(19) United States
(12) Patent Application Publication
(10) Pub. No.: US 2016/0074517 A1
(43) Pub. Date: Mar. 17, 2016

(54) AQUEOUS FORMULATIONS OF SPARINGLY WATER-SOLUBLE ACTIVE INGREDIENTS COMPRISING AS SOLUBILIZER A MIXTURE OF POLOXAMERS DIFFERING IN HYDROPHOBICITY

(71) Applicant: BASF SE, Ludwigshafen (DE)

(72) Inventors: Viet Nguyen-Kim, Heidelberg (DE); Ann-Kathrin Marguerre, Heidelberg (DE); Murat Mertoglu, Ludwigshafen (DE); Eve Leonie Leinenbach, Mannheim (DE)

(21) Appl. No.: 14/888,951

(22) PCT Filed: Apr. 25, 2014
(86) PCT No.: PCT/EP2014/058435

§ 371 Date: Nov. 4, 2015

(30) Foreign Application Priority Data
May 6, 2013 (EP) 13166662.0

(51) Int. Cl. A61K 47/34; A61K 31/216; A01N 25/30; A01N 43/56; A01N 25/02; A61K 9/08

(52) U.S. Cl. CPC A61K 47/34; A01N 25/02; A61K 9/08; A01N 25/30; A01N 43/56; A61K 31/216

(57) ABSTRACT

Use of a mixture

a) of a hydroxy-terminated polyether of the block structure (A) (EO)x1′-(PO)y1-(EO)x1″, where y1 is 40 to 70 and (x1′+x1″) is 30-60 and (x1′+x1″)/y1 is less than 1 (component A), and

b) of a hydroxy-terminated polyether of the block structure (B) (EO)x2′-(PO)y2-(EO)x2″, where y2 is 15 to 55 and (x2′+x2″) is 2-50 and (x2′+x2″)/y2 is less than 1 (component B), where (x1′+x1″)/y1 is >(x2′+x2″)/y2, for solubilizing sparingly water-soluble active ingredients, which have a solubility in water of not more than 5 g/l at 25° C. and 0.1013 MPa, in aqueous formulations.

The present invention relates to the use of a mixture
a) of a hydroxy-terminated polyether of the block structure (A) (EO)x1′-(PO)y1-(EO)x1″, where y1 is 40 to 70 and (x1′+x1″) is 30-60 and (x1′+x1″)/y1 is less than 1 (component A), and
b) of a hydroxy-terminated polyether of the block structure (B) (EO)x2′-(PO)y2-(EO)x2″, where y2 is 15 to 55 and (x2′+x2″) is 2-50 and (x2′+x2″)/y2 is less than 1 (component B),
and
where (x1′+x1″)/y1 is >(x2′+x2″)/y2,
   for solubilizing sparingly water-soluble active ingredients in aqueous formulations. Furthermore, the invention relates to corresponding aqueous active ingredient formulations.

Many active ingredients, especially bioactive ingredients, are supposed to develop their effect in aqueous media. On the other hand, many of these active ingredients are hydrophobic, meaning that they can only develop inadequate solubility and thus inadequate bioavailability in an aqueous medium.

In order to overcome this disadvantage, the active ingredients are therefore often used as micellar systems in which the active ingredient is present in solubilized form by means of surface-active substances.

Amphiphilic polymers are often used as solubilizers, especially for agrochemical or pharmaceutical formulations. Polymers that are often used here are the poloxamers. Poloxamer is the non-proprietary name for triblock polyether copolymers of ethylene oxide and propylene oxide units.

Poloxamers are triblock polymers which have an A-B-A block structure. (A=Polyethylene oxide; B=polypropylene oxide)

U.S. Pat. No. 5,672,564 discloses the use of poloxamers in combination with further surfactants.

US 20060013871 discloses the improvement in the solubility of ibuprofen by coating the ibuprofen with a poloxamer.

US-A 20110008266 or US 20070141143, for example, likewise describe the use of poloxamers in pharmaceutical formulations.

WO 2006002984 discloses liquid pesticide formulations which comprise nonionic polyether block copolymers as solubility promoters.

US-A 20070015834 discloses mixtures of poloxamers and polyethylene glycol for formulating fenofibrate.

WO 03/017976 discloses the use of synergistic mixtures of poloxamers as solubilizers for pharmaceutical active ingredients. Mixtures of Poloxamer 407 with the block structure (EO)₁₀₁-(PO)₅₆-(EO)₁₀₁ and Poloxamer 188 with the block structure (EO)₈₀-(PO)₂₇-(EO)₈₀ are specifically described. The poloxamers used therein have relatively high ethylene oxide fractions.

A disadvantage of this is that the solubilizing effect of known mixtures is often not satisfactorily pronounced.

Although PO-rich surfactants on their own often have a solubilizing effect, in relatively large concentrations they lead to a considerable increase in the viscosity of the solution, meaning that they are unsuitable for aqueous formulations as regards processing. In relatively small concentrations, however, the solubilizing effect is often not pronounced enough in order to be of practical use.

Mixtures with EO-richer poloxamers sometimes have a tendency towards gelation, which is likewise undesirable for administration in the form of aqueous formulations.

It was an object of the present invention to provide solubilizers which help to avoid the disadvantages of the prior art.

Accordingly, use of a mixture
a) of a hydroxy-terminated polyether of the block structure (A) (EO)x1′-(PO)y1-(EO)x1″, where y1 is 40 to 70 and (x1′+x1″) is 30-60 and (x1′+x1″)/y1 is less than 1 (component A), and
b) of a hydroxy-terminated polyether of the block structure (B) (EO)x2′-(PO)y2-(EO)x2″, where y2 is 15 to 55 and (x2′+x2″) is 2-50 and (x2′+x2″)/y2 is less than 1 (component B), where (x1′+x1″)/y1 is >(x2′+x2″)/y2, for solubilizing sparingly water-soluble active ingredients in aqueous formulations has been found. Preferably, y2 is 15 to 35.

Furthermore, aqueous active ingredient formulations comprising a sparingly water-soluble active ingredient and
a) 2 to 20% by weight of a hydroxy-terminated polyether of the block structure (A) (EO)x1′-(PO)y1-(EO)x1″, where y1 is 40 to 70 and (x1′+x1″) is 30-60 and (x1′+x1″)/y1 is less than 1 (component A), and
b) 1 to 40% by weight of a hydroxy-terminated polyether of the block structure (B) (EO)x2′-(PO)y2-(EO)x2″, where y2 is 15 to 55 and (x2′+x2″) is 2-50 and (x2′+x2″)/y2 is less than 1 (component B),
   where (x1′+x1″)/y1 is >(x2′+x2″)/y2, and where the weight fractions are based on the total weight of the aqueous formulation, have been. Preferably, y2 is 15 to 35, found.

Furthermore, it is the case that for components A and B the quotient (x1′+x1″)/y1 is greater than (x2′+x2″)/y2. The EO:PO ratio of component A should thus be greater than the EO:PO ratio of component B.

According to the invention, the term “sparingly water-soluble active ingredient” means that at 25° C. and 0.1013 MPa the solubility in water of the active ingredient is not more than 5 g/l, in particular not more than 1 g/l, very particularly not more than 0.1 g/l.

HLB value means Hydrophilic-Lipophilic-Balance. The HLB value can be calculated for nonionic surfactants according to the following formula:

HLB=20×(1−M₁/M), where M₁ is the molecular weight fraction of the lipophilic molecular moiety and M is the total molecular weight. In the present case, M₁ is the polypropylene oxide fraction (PO).

Poloxamers is the term used to refer to hydroxy-terminated triblock copolymers of the block structure A-B-A, where A is ethylene oxide units (EO) and b is propylene oxide units PO. As component A, polyethers of the block structure (A) (EO)x1′-(PO)y1-(EO)x1″, where y1 is 40 to 70, preferably 45 to 60, and (x1′+x1″) is 30-60, preferably 30 to 50, and (x1′+x1″)/y1 is less than 1 are used as triblock copolymers. Particularly preferably, x1′=x1″. In the corresponding polyethers, the ratio of the total content of EO units to PO units is 1:1.05 to 1:2, preferably 1:1.05 to 1:1.

The polyethers are hydroxy-terminated and have the general formula HO(CH₂—CH₂O)_{x1′}—(CH(CH₃)—CH₂O)_{y1}—(CH₂—CH₂O)_{x1″}—H. The HLB value can be 6 to 8.

Suitable polyether triblock copolymers are for example

HO(CH₂—CH₂O)₂₅—(CH(CH₃)—CH₂O)₅₆—(CH₂—CH₂O)₂₅—H,

HO(CH₂—CH₂O)₁₆—(CH(CH₃)—CH₂O)₅₆—(CH₂—CH₂O)₁₆—H,

HO(CH₂—CH₂O)₂₁—(CH(CH₃)—CH₂O)₄₇—(CH₂—CH₂O)₂₁—H.

Mixtures of component A can also be used.

Corresponding poloxamers are commercially available, for example under the trade name Pluronic® PE. A particularly preferred poloxamer A is the compound of the formula HO(CH₂—CH₂O)₂₅—(CH(CH₃)—CH₂O)₅₆—(CH₂—CH₂O)₂₅—H commercially available as Pluronic®-PE type 10400.

Polyethers of the block structure (B) (EO)x2′-(PO)y2-(EO)x2″, where y2 is 15 to 55, preferably 15 to 35, and (x2′+x2″) is 2-50, preferably 10 to 50, and (x2′+x2″)/y2 is less than 1 are used as component B. Particularly preferably, x2′=x2″. The ratio of the total content of EO units to PO units (EO:PO) is 1:1.1 to 1:14, preferably 1:1.5 to 1:8, particularly preferably 1:1.5 to 1:3. The HLB value is 0 to 6, preferably 2 to 6.

The polyethers are hydroxy-terminated and have the general formula HO(CH₂—CH₂O)_{x2′}—(CH(CH₃)—CH₂O)_{y2}—(CH₂—CH₂O)_{y2″}—H.

Suitable components B are for example

HO(CH₂—CH₂O)₈—(CH(CH₃)—CH₂O)₄₇—(CH₂—CH₂O)₈—H

HO(CH₂—CH₂O)₁—(CH(CH₃)—CH₂O)₁₆—(CH₂—CH₂O)₁—H

HO(CH₂—CH₂O)₂—(CH(CH₃)—CH₂O)₁₆—(CH₂—CH₂O)₂—H

HO(CH₂—CH₂O)₂—(CH(CH₃)—CH₂O)₃₀—(CH₂—CH₂O)₂—H

HO(CH₂—CH₂O)₄—(CH(CH₃)—CH₂O)₅₆—(CH₂—CH₂O)₄—H

HO(CH₂—CH₂O)₅—(CH(CH₃)—CH₂O)₁₉—(CH₂—CH₂O)₅—H

HO(CH₂—CH₂O)₅—(CH(CH₃)—CH₂O)₃₀—(CH₂—CH₂O)₅—H

Mixtures of components B can also be used.

Components B are also commercially available, for example under the trade name Pluronic® PE. Particularly preferred poloxamers A are the compounds of the formulae HO(CH₂—CH₂O)₅—(CH(CH₃)—CH₂O)₁₉—(CH₂—CH₂O)₅—H and HO(CH₂—CH₂O)₅—(CH(CH₃)—CH₂O)₃₀—(CH₂—CH₂O)₅—H commercially available as Pluronic®-PE grades 4300 and 6200.

The quantitative ratio of component A to component B can be 1:1 to 1:10, preferably 1:2 to 1:8.

Based on the total amount of aqueous formulation, the component A can be used in amounts of from 2.5 to 15% by weight, preferably 5 to 10% by weight.

According to the invention, the mixture of components A and B is used for solubilizing sparingly water-soluble active ingredients in aqueous formulations.

Sparingly soluble active ingredients may be pharmaceutical, agrochemical or cosmetic active ingredients or food supplements or animal feed additives. Pharmaceutical active ingredients can originate from all areas of indication. Agrochemical active ingredients can for example be pesticides such as insecticides, fungicides or herbicides or seed treatments. Cosmetic active ingredients can for example be photoprotective compositions, cosmetic oils or pigments. Food supplements may be fat-soluble vitamins, carotenoids, ubiquinone or oily substances.

To use the aqueous formulations, the conventional auxiliaries acceptable for the particular application can furthermore be added.

Suitable additional auxiliaries are: thickeners, further surfactants, stabilizers, antioxidants, biocides, defoamers or dyes. Furthermore, organic solvents can also additionally be present.

It is also advantageous that according to the invention not only can a good solubilization of the active ingredient be achieved, but also the viscosity of the aqueous solutions can be kept low and gelation of the aqueous formulations can be prevented.

### EXAMPLES

General procedure for the preparation of the aqueous formulations:

The data in % by weight in the tables below refer to the total amount of the aqueous formulation.

Components A and B in the amounts stated below in each case and also 2% by weight of active ingredient were dissolved in water (at 20° C. and ambient pressure).

The sample was stirred at 20° C. for 24 h (magnetic stirrer 500 rpm) and then filtered through an injection filter (PVDF filter membrane, 0.45 μm pore size).

The active ingredient quantification takes place via UV/Vis spectroscopy. For this, the samples were diluted with an organic solvent in which the active ingredient dissolves to at least 10 000 ppm (1% by weight) (ethanol).

The poloxamers used were the following compounds.

Component A:

Pluronic® PE 10400: HO(CH₂—CH₂O)₂₅—(CH(CH₃)—CH₂O)₅₆—(CH₂—CH₂O)₂₅—H

Components B:

Pluronic® PE 4300: HO(CH₂—CH₂O)₅—(CH(CH₃)—CH₂O)₁₉—(CH₂—CH₂O)₅—H

Pluronic® PE 6200: HO(CH₂—CH₂O)₅—(CH(CH₃)—CH₂O)₃₀—(CH₂—CH₂O)₅—H.

For comparison:

The solubility of fenofibrate in a 5% strength by weight aqueous solution of PE 10400 (component A) is 1089 ppm.

The solubility of fenofibrate in a 5% strength by weight aqueous solution of PE 4300 (component B) is 3 ppm.

The solubility of fenofibrate in a 5% strength by weight aqueous solution of PE 6200 (component B) is 4 ppm.

The solubility of fenofibrate in pure water is likewise only 3 ppm.

Since the components B on their own exhibit no solubilizing effect compared to PE 10400, their solubilization effect for a 5% strength by weight solution was standardized to zero. By contrast, the solubilizing effect of a 5% strength by weight aqueous solution of PE10400 as individual component was stipulated as standard with the value 1 and the solubilizing effect of the mixtures was determined against this standard.

TABLE I

| | | | Active | | Increase in |
|---|---|---|---|---|---|
| | Pluronic | Pluronic PE | ingredient/% | Water | solubilization |
| | PE10400 | 6200 | by weight | % by | compared with |
| | % by weight | % by weight | (fenofibrate) | weight | standard |
| (a) | 0 | 5 | 2 | 93 | 0 |
| (b) | 5 | 0 | 2 | 93 | 1 (standard) |
| (c) | 5 | 5 | 2 | 88 | 1.3 |
| (d) | 5 | 10 | 2 | 83 | 1.6 |
| (e) | 5 | 15 | 2 | 78 | 2.1 |
| (f) | 5 | 20 | 2 | 73 | 3.5 |
| (g) | 5 | 30 | 2 | 63 | 6.2 |
| (h) | 5 | 40 | 2 | 58 | 12.9 |

TABLE II

| | | | Active | | Increase in |
|---|---|---|---|---|---|
| | Pluronic | Pluronic PE | ingredient/% | Water | solubilization |
| | PE10400 | 4300 | by weight | % by | compared with |
| | % by weight | % by weight | (fenofibrate) | weight | standard |
| (a) | 5 | 5 | 2 | 88 | 1.2 |
| (b) | 5 | 10 | 2 | 83 | 1.3 |
| (c) | 5 | 15 | 2 | 78 | 1.6 |
| (d) | 5 | 20 | 2 | 73 | 1.9 |
| (e) | 5 | 30 | 2 | 63 | 3.6 |
| (f) | 5 | 40 | 2 | 58 | 7.8 |

For comparison:

The solubility of fluxapyroxad in a 5% strength by weight aqueous solution of PE 10400 (component A) is 710 ppm.

The solubility of fluxapyroxad in a 5% strength by weight aqueous solution of PE 4300 (component B) is 28 ppm.

The solubility of fluxapyroxad in a 5% strength by weight aqueous solution of PE 6200 (component B) is 31 ppm.

The solubility of fluxapyroxad in pure water is only 5 ppm.

The solubilizing effect of a 5% strength by weight aqueous solution of PE 10400 was again selected as standard 1.

TABLE III

| | Pluronic | Pluronic | Active | | Increase in |
|---|---|---|---|---|---|
| | PE10400 | PE 6200 | ingredient/% | | solubilization |
| | % by | % by | by weight | Water % | compared with |
| | weight | weight | (fluxapyroxad) | by weight | standard |
| (a) | 5 | 5 | 2 | 88 | 1.4 |
| (b) | 5 | 10 | 2 | 83 | 2.0 |
| (c) | 5 | 15 | 2 | 78 | 2.7 |
| (d) | 5 | 20 | 2 | 73 | 3.8 |
| (e) | 5 | 25 | 2 | 68 | 5.2 |
| (f) | 5 | 30 | 2 | 63 | 8.9 |
| (g) | 5 | 40 | 2 | 58 | 14.5 |

TABLE IV

| | Pluronic | Pluronic | Active | | Increase in |
|---|---|---|---|---|---|
| | PE10400 | PE 4300 | ingredient/% | | solubilization |
| | % by | % by | by weight | Water % | compared with |
| | weight | weight | (fluxapyroxad) | by weight | standard |
| (a) | 5 | 5 | 2 | 88 | 1.3 |
| (b) | 5 | 10 | 2 | 83 | 1.5 |
| (c) | 5 | 15 | 2 | 78 | 1.9 |
| (d) | 5 | 20 | 2 | 73 | 2.4 |
| (e) | 5 | 25 | 2 | 68 | 3.4 |

In order to show that the viscosity of the mixture is advantageous compared with the viscosity of the pure solution of component A in water, the viscosities of the aqueous solutions were determined. The viscosities were measured using a rheometer from Anton Paar (model: Physics MCR-301). The measurement temperature was 20° C. The measurement was carried out at ambient pressure.

TABLE V

| Pluronic ® PE | Viscosity of PE |
|---|---|
| 10400 | 10400 solution |
| 5% | 1.85 |
| 10% | 3.22 |
| 20% | 14.35 |
| 30% | does not dissolve |
| | completely; gel-like |
| Pluronic ® mixture | |
| of PE 10400 (5%) | viscosity of |
| with % PE 6200 | Pluronic ® mixture |
| 5% | 2.69 |
| 10% | 4.37 |
| 20% | 9.38 |

% = % by weight

1.-17. (canceled)

18. An aqueous active ingredient formulation comprising a sparingly water-soluble active ingredient, which has a solubility in water of not more than 5 g/l at 25° C. and 0.1013 MPa, and, as solubilizer for the sparingly water-soluble active ingredient, a mixture
a) of a hydroxy-terminated polyether of the block structure (A) (EO)x1′-(PO)y1-EO)x1″, where y1 is 40 to 70 and (x1′+x1″) is 30-60 and (x1′+x1″)/y1 is less than 1 (component A), and
b) of a hydroxy-terminated polyether of the block structure (B) (EO)x2′-(PO)y2-(EO)x2″, where y2 is 15 to 55 and (x2′+x2″) is 2-50 and (x2′+x2″)/y2 is less than 1 (component B), and where (x1′+x1″)/y1 is >(x2′+x2″)/y2.

19. The aqueous formulation according to claim 18, where a polyether of the block structure (A) (EO)x1′-(PO)y1-(EO)x1″ where y=45 to 60 is used as component A.

20. The aqueous formulation according to claim 18, where a polyether of the block structure (A) (EO)x1′-(PO)y1-(EO)x1″ where (x1′+x1″)=30 to 50 is used as component A.

21. The aqueous formulation according to claim 18, where a polyether where x1′=x1″ is used as component A.

22. The aqueous formulation according to claim 18, where polyethers in which the ratio of the total content of EO units to PO units is 1:1.05 to 1:2 are used as components A.

23. The aqueous formulation according to claim 18, where polyethers in which the ratio of the total content of EO units to PO units is 1:1.05 to 1:1 are used as components A.

24. The aqueous formulation according to claim 18, where a polyether selected from the group consisting of
HO(CH₂—CH₂O)₂₅—(CH(CH₃)—CH₂O)₅₆—(CH₂—CH₂O)₂₅—H,
HO(CH₂—CH₂O)₁₆—(CH(CH₃)—CH₂O)₅₆—(CH₂—CH₂O)₁₆—H, and
HO(CH₂—CH₂O)₂₁—(CH(CH₃)—CH₂O)₄₇—(CH₂—CH₂O)₂₁—H is used as component A.

25. The aqueous formulation according to claim 18, where the polyether of the formula HO(CH₂—CH₂O)₂₅—(CH(CH₃)—CH₂O)₅₆—(CH₂—CH₂O)₂₅—H is used as component A.

26. The aqueous formulation according to claim 18, where a polyether of the block structure (B) (EO)x2′-(PO)y2-(EO)x2″ where (x2′+x2″)=10-50 is used as component B.

27. The aqueous formulation according to claim 18, where a polyether of the block structure (B) (EO)x2′-(PO)y2-(EO)x2″ where x2′=x2″ is used as component B.

28. The aqueous formulation according to claim 18, where a component B with an HLB value of from 2 to 6 is used.

29. The aqueous formulation according to claim 18, where polyethers in which the ratio of the total content of EO units to PO units is 1:1.1 to 1:14 are used as components B.

30. The aqueous formulation according to claim 18, where polyethers in which the ratio of the total content of EO units to PO units is 1:1.5 to 1:8 are used as components B.

31. The aqueous formulation according to claim 18, where a polyether selected from the group consisting of
HO(CH₂—CH₂O)₈—(CH(CH₃)—CH₂O)₄₇—(CH₂—CH₂O)₈—H,
HO(CH₂—CH₂O)₁—(CH(CH₃)—CH₂O)₁₆—(CH₂—CH₂O)₁—H,
HO(CH₂—CH₂O)₂—(CH(CH₃)—CH₂O)₁₆—(CH₂—CH₂O)₂—H,
HO(CH₂—CH₂O)₂—(CH(CH₃)—CH₂O)₃₀—(CH₂—CH₂O)₂—H,
HO(CH₂—CH₂O)₄—(CH(CH₃)—CH₂O)₅₆—(CH₂—CH₂O)₄—H,
HO(CH₂—CH₂O)₅—(CH(CH₃)—CH₂O)₁₉—(CH₂—CH₂O)₅—H, and
HO(CH₂—CH₂O)₅—(CH(CH₃)—CH₂O)₃₀—(CH₂—CH₂O)₅—H is used as component B.

32. The aqueous formulation according to claims 18, where
HO(CH₂—CH₂O)₅—(CH(CH₃)—CH₂O)₁₉—(CH₂—CH₂O)₅—H or
HO(CH₂—CH₂O)₅—(CH(CH₃)—CH₂O)₃₀—(CH₂—CH₂O)₅—H or a mixture thereof is used as component B.

33. The aqueous formulation according to claim 18, where a quantitative ratio of component A to component B is 1:1 to 1:10.

34. The aqueous formulation according to claim 18, where a quantitative ratio of component A to component B is 1:2 to 1:8.

35. The aqueous formulation according to claim 18, comprising a pharmaceutical active ingredient, an agrochemical active ingredient, a cosmetic active ingredient, or a food supplement as the sparingly water-soluble active ingredient.

36. (canceled)

37. (canceled)

38. (canceled)

39. The aqueous formulation according to claim 18, additionally comprising one or more thickeners, organic solvents, further surfactants, stabilizers, antioxidants, biocides, defoamers, or dyes.

40. A method of solubilizing a sparingly water-soluble action ingredient in an aqueous formulation comprising the use of a mixture
a) of a hydroxy-terminated polyether of the block structure (A) (EO)x1′-(PO)y1-(EO)x1″, where y1 is 40 to 70 and (x1′+x1″) is 30-60 and (x1′+x1″)/y1 is less than 1 (component A), and
b) of a hydroxy-terminated polyether of the block structure (B) (EO)x2′-(PO)y2-(EO)x2″, where y2 is 15 to 55 and (x2′+x2″) is 2-50 and (x2′+x2″) /y2 is less than 1 (component B), where (x1′+x1″)/y1 is >(x2′+x2″)/y2, wherein the sparingly water-soluble active ingredient has a solubility in water of not more than 5 g/l at 25° C. and 0.1013 MPa.
